(19) United States
(12) Patent Application Publication
(10) Pub. No.: US 2008/0004571 A1
(43) Pub. Date: Jan. 3, 2008

(54) EXPANDABLE INTRODUCER SHEATH

(75) Inventor: Laveille Kao Voss, Belmont, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Redwood City, CA (US)

(21) Appl. No.: 11/427,308

(22) Filed: Jun. 28, 2006

(51) Int. Cl. A61M 5/178

(52) U.S. Cl. 604/167.01

(57) ABSTRACT

An introducer sheath. An introducer sheath is formed as a unitary member using an injection molding process or a co-extrusion process. The introducer sheath includes a hub portion that is integrally formed with a sheath portion. The sheath portion may include a second material integrally formed with the sheath portion. The second material may aid is splitting of the sheath. The sheath portion may alternatively have a geometric pattern formed on an inner wall of the sheath to aid is splitting of the sheath.

### CROSS-REFERENCE TO RELATED APPLICATIONS

This application relates to U.S. patent application Ser. No. ______, filed Jun. 28, 2006, and entitled “Modular Introducer and Exchange Sheath” (Attorney Docket No. 16497.12.1) and U.S. patent application Ser. No. ______, filed Jun. 28, 2006, and entitled “Expandable Introducer Sheath” (Attorney Docket No. 16497.13.1), the disclosures of which are incorporated herein by this reference.

### BACKGROUND OF THE INVENTION

1. The Field of the Invention

The present invention relates generally to medical devices and methods. More particularly, embodiments of the invention relate to expandable medical devices, such as introducer sheaths, for use during medical procedures.

2. The Relevant Technology

A wide variety of devices have been developed for medical use. One such device is an introducer sheath that facilitates access to body lumen at an access site. Conventionally, introducer sheaths are formed of three or more components that require assembly: a sheath portion, a hub, and a hemostasis valve disposed within the hub. A suitable example of such an assembly is shown in U.S. Pat. No. 5,807,350, which depicts an introducer sheath having a construction similar to that described above, the entirety of which is hereby incorporated by reference.

In practice, introducer sheaths are often used to access a vessel or artery to allow a surgical or medical procedure to be performed. The introducer sheath is generally inserted into a patient's vasculature using the modified Seldinger technique. In the Seldinger technique, a needle is first inserted into the vessel and then followed by a guide wire through the needle. Next, the needle is removed and a sheath/dilator combination is advanced over the guide wire. The sheath/dilator expands the puncture in the vessel to a size suitable to receive the distal end of an introducer sheath. After the distal end of the sheath is disposed within the vessel, the dilator and guide wire are removed, thereby allowing access to the vessel lumen through the introducer sheath.

There are an increasing number of medical procedures that can be performed using sheaths. Medical procedures such as angioplasty, stenting, and intraaortic therapy, are examples of procedures that can include the use of introducer sheaths. In particular, the medical devices (e.g., catheters, balloon pumps) used in these procedures are introduced through the sheath.

Some of these procedures unfortunately require removal of the sheath earlier than desired. For example, intraaortic balloon pump therapy for ventricular insufficiency is often performed using a sheath. In this procedure, a sheath is inserted into the femoral artery. Next, a balloon pump is introduced into the patient's vasculature through the sheath and then guided to the aortic arch region. Once the balloon pump is properly positioned in the arch region, it is left in place until the ventricular insufficiency is improved to an acceptable level, which may take days.

In this procedure, the balloon pump is being inflated and deflated at a rate that typically matches the heart rate. Thus, the balloon pump is usually inflated during ventricular diastole and deflated during ventricular systole. As a result of the use of the balloon pump for the intraaortic therapy, the balloon pump will be larger in size compared to when it was initially inserted through the sheath. As a result of the increased size, removal of the balloon pump also requires the removal of the sheath since the enlarged balloon pump cannot fit inside of the sheath tubing. One of the disadvantages of removing the sheath along with the balloon pump is that the opportunity to close the vessel with any vessel closure device through the sheath is lost.

There is therefore a need for a new introducer sheath to accommodate removal and/or insertion of devices that change in size or that do not work with conventional sheaths. There is also a need for sheaths that have lower manufacturing costs.

### BRIEF SUMMARY OF THE INVENTION

These and other limitations are overcome by embodiments of the invention, which relates to medical devices and in particular to expandable introducer sheaths. Embodiments of the invention provide several designs and methods of manufacture of an expandable introducer sheath. One embodiment is an introducer sheath formed as a unitary device using, for example, and injection molding process or a co-extrusion process.

In one configuration of an expandable introducer sheath, one or more materials are used to form the sheath. At least one of the materials provides elasticity. The elasticity enables the sheath to expand, thereby accommodating the introduction and/or removal of medical devices that could not previously be accommodated.

In one configuration, the introducer sheath includes a hub portion and a tubular portion. A valve member (such as a hemostasis valve) can be disposed into the hub portion either during the molding process or after the initial molding process. The hemostasis valve can be retained either by an additional element such as a cap or through an element formed during the molding process or during a subsequent molding process.

The tubular portion can include a sheath portion and an elastic portion, which can be formed as a unitary portion through injection molding or co-extrusion processes, for example. The elastic portion of the sheath may be an elastomer that is integrated with another material in the sheath portion that provides rigidity and/or prevents a lumen of the tubular portion from collapsing. When a medical device is withdrawn, for example, the elastic portion can expand thereby permitting the medical device to be withdrawn without splitting the introducer sheath. Advantageously, this enables the use of a subsequent medical device, such as a vessel closure device, to be introduced through the intact sheath, whether or not the vessel closure device splits or cuts the introducer sheath during use.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of an exemplary embodiment of an introducer sheath in accordance with the present invention;

FIG. 2 illustrates a cross sectional view of one embodiment of the introducer sheath in FIG. 1;

FIG. 3 illustrates a cross sectional view of another embodiment of the introducer sheath in FIG. 1;

FIG. 4 illustrates a cross sectional view of yet another embodiment of the introducer sheath in FIG. 1;

FIG. 5 illustrates a cross sectional view of another embodiment of the introducer sheath in FIG. 1;

FIG. 6A illustrates an introducer sheath prior to insertion of a medical device;

FIG. 6B illustrates an introducer sheath prior to removal of a medical device that has changed size during use;

FIG. 6C illustrates an embodiment of the introducer sheath during removal of the medical device that has changed size during use; and

FIG. 6D illustrates the use of a vessel closure device that is introduced through the sheath after the medical device illustrated in FIGS. 6A and 6B has been withdrawn.

### DETAILED DESCRIPTION OF THE PREFERRED EMBODIMENTS

Embodiments of the invention relate to a device that is expandable to cooperate with medical devices that may have become enlarged in dimension during use. For instance, in one configuration, the device can be an expandable introducer sheath that can accommodate removal of enlarged medical devices without removing the introducer from the delivery site. As such, the sheath or at least a portion of the introducer sheath can expand to accommodate the introduction and/or removal of medical devices that could not ordinarily be accommodated in conventional sheaths. At the same time, the sheath can be formed to have desirable stiffness, kink resistance, and/or flexibility for insertion and positioning in at least a portion of a body lumen. Embodiments of the sheath are depicted in the drawings, which are not necessarily to scale and are not intended to limit the scope of the invention. It will be understood that the benefits of the present invention are not limited to application with an introducer sheath. Rather, other medical devices may be modified based upon the teaching contained herein such that they to can provide the functionality of accommodating removal of enlarged medical devices.

Turning to the introducer sheath in accordance with the present invention, the sheath will be described herein as having portions or members, though it shall be understood that the sheath as described herein can be formed as a unitary unit, formed, by way of example, using a co-extrusion process or an injection molding process, or a sheath fabricated from the assembly of separate parts. As such, the various members or portions are used herein for clarification only and in no way limit the applicability of description herein to other configurations of the sheath and/or medical devices.

Generally stated, an exemplary introducer sheath can include a hub member or portion having a proximal end and a distal end. The proximal end of the sheath can be configured to receive a flexible valve member therein. The sheath can further include an elongated tubular member or portion generally extending from the distal portion of the hub portion. The elongated tubular portion, in one configuration, can be generally axially aligned with an axis of the hub portion, with the lumen of the tubular portion being aligned with a lumen of the hub portion. Alternatively, the lumen of the tubular portion can be aligned with a lumen of the hub portion, whether or not axially aligned. The aligning of the lumens can occur during manufacture, such as when the hub portion and the sheath are formed as a single integrated unit or when separate components are joined together. In one embodiment, the tubular portion is configured to expand while still providing the necessary stiffness and/or kink resistance to the sheath.

An introducer sheath or portions thereof can be formed using one or more materials. Typically, the materials used in forming the introducer sheath are medical grade synthetic materials or plastics. Exemplary materials may include, but are not limited to, flexible PVC, polyurethane, silicone, liner low-density polyethylene (“LLDPE”), polyethylene, high density polyethylene, (“DUPE”), polyethylene-lined ethylvinyl acetate (“PE-EVA”), polypropylene, latex, thermoplastic rubber, polytetrafluorethylene (PTFE), expandable polytetrafluorethylene (ePTFE), fluroethylene-propylene (FEP), perfluoralkoxy (PFA), ethylene-tetrafluoroethylene-copolymer (ETFE), ethylene-chlorotrifluoroethylene (ECTFE), polychloro-trifluoroethylene (PCTFE), polyimide (PI), polyetherimide (PEI), polyetherketone (PEEK), polyamide-imide (PAI), other fluoropolymers, and the like.

Exemplary materials used in the sheath or a portion of the sheath can also include elastomers or thermoplastic elastomers. Examples of elastomers include, but are not limited to, natural rubber, silicone rubber, polyurethane rubber, polybutadiene, polyisoprene, chlorosulfonated polyethylene, polysulfide rubber, epichlorohydrin rubber, resilin, ethylene propylene rubber, and the like or any combination thereof These materials provide the elasticity that enable the sheath to expand and/or contract to accommodate the removal/insertion of a medical device as required. Other materials that can be used can include, but are not limited to, dip coated type silicones.

In other embodiments, the materials suitable for use in an introducer sheath are configured to have chemical resistance, crack resistance, no toxicity, Food and Drug Administration (“FDA”) compliance, non-electrically conductive, dimensional stability, and/or be sterilized by ethylene oxide, gamma radiation, autoclave, UV light, ozone, and the like.

In addition, the selection of materials for a particular sheath can depend on a variety of factors that include, but are not limited to, a particular stiffness and/or flexibility of the sheath or any portion of the sheath, including the desired column stiffness and strength to enable insertion of the sheath, a particular shear or split strength for the sheath or any portion of the sheath, the ability to resist kinking, and the like. For example, the material used for the tubular portion of the introducer sheath may be selected based on shear strength or how easily it can be split. Further, certain features of the sheath may be formed to enhance certain characteristics. For example, a strain relief portion may be formed so as to resist kinking while the elongated tubular portion may be formed to facilitate splitting.

When more than one material is used to form the sheath or to form specific portions of the introducer sheath, the materials may be selected, in addition to the factors identified herein, on a bond strength between the materials or on the elasticity of a particular material. The bond strength, for example, may have an impact on the splitability of the sheath or of a portion of the sheath. The bond strength may also affect the ability of the sheath to expand without splitting.

When an elastomer is used in the sheath or a portion of the sheath, the elasticity of the elastomer enables the sheath or a portion of the sheath to at least partially deform, resiliently deform, or elastically expand as needed to accommodate a medical device and then return or substantially return to its configuration prior to deforming or expanding. Advantageously, the ability to deform and/or expand permits a device, such as an expanded or expandable balloon, to be withdrawn through the sheath without removing the sheath, for example from a patient's vasculature. This maintains access to the patient's vasculature without the difficulty of inserting another sheath or medical device through the puncture site. Further, maintaining the introducer sheath in place allows a physician or technician to insert one or more additional medical devices, such as a vessel closure device, using the introducer sheath.

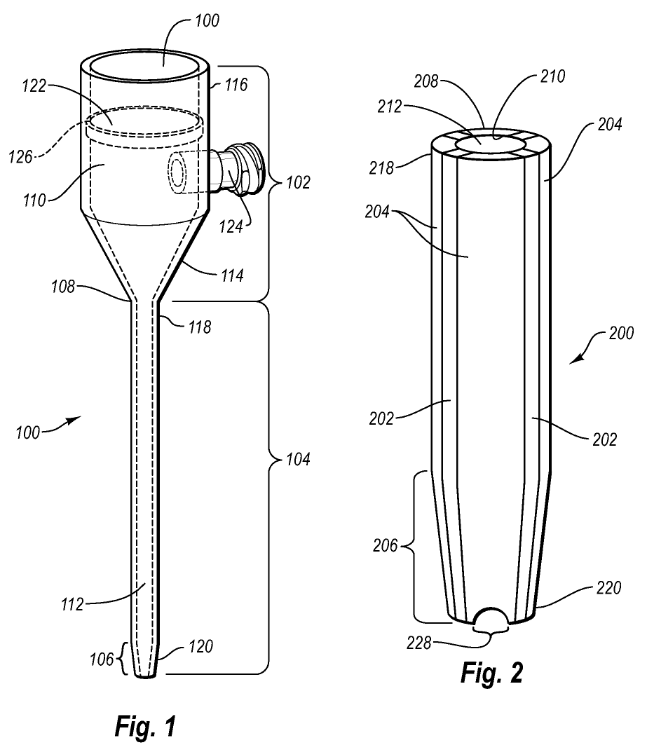

Referring now to FIG. 1, there is shown an exemplary embodiment of an introducer sheath 100. The introducer sheath 100 can include a hub portion 102, which can include a proximal end 116 and a distal end 114, and a tubular portion 104. Extending from the proximal end 116 toward the distal end 114 is a lumen 110. This lumen 110 can cooperate with a medical device (not shown), such as a vessel closure device, insertable therethrough. In the illustrated configuration, the lumen 110 tapers or transitions from one cross-sectional configuration to another cross-sectional configuration near the distal end 114 to meet or intersect with a lumen 112 of the tubular portion 104. It will be understood that the lumen 110 can have a generally uniform cross-section along its length rather than tapering at its distal end. More generally, the lumen 110 can include one or more transitional portions based upon the desired configuration and use with other medical devices.

The elongated tubular portion 104 of the introducer sheath 100 can extend from the distal end 114 of the hub portion 102. The tubular portion 104 can include a distal end 120 and a proximal end 118. The proximal end 118 can be integrally formed with the distal end 114 of the hub portion 102 or can be mounted or coupled to the distal end 114 through a friction fit, mechanical bonding, adhesives, thermal or chemical bonding, combinations thereof or other manufacturing technique usable to mount, couple or attach two medical components. The distal end 120 of the tubular portion 104 can optionally include a tapered portion 106 to facilitate insertion into a body lumen. This tapered portion 106 may be formed during or after the initial forming process of the introducer sheath 100. For instance, when the introducer sheath 100 is formed through a molding or extrusion process, the tapered portion 106 can be formed as part of this process. Alternatively, the tapered portion 106 may be formed by heat forming, grinding or other known methods that result in a thinner wall thickness following the above-described molding or extrusion process or as part of a milling, machining, or similar process.

Optionally disposed at the transition between the hub portion 102 and the tubular portion 104 is a strain relief portion 108. The strain relief portion 108 would be disposed adjacent the distal end 114 of the hub portion 102 and adjacent the proximal end 118 of the elongate tubular portion 104. The strain relief 108 would be configured to provide additional support to the proximal end 118 of the elongated shaft 104 to prevent kinking at the transition between the proximal end 118 of the elongated member 104 and the distal end 114 of the hub portion 102. In one embodiment, the strain relief portion 108 can be formed by gradually increasing a thickness of tubular portion 104 at the transition between the tubular portion 104 and the hub portion 102. In other configurations, the strain relief portion 108 can include webs, extensions, or other internal or external structures to increase the strength and/or stiffness of the introducer sheath 100 at the hub portion/tubular portion transition.

The tubular portion 104 of the introducer sheath can be expandable. More specifically, in the illustrated configuration of FIG. 1, the tubular portion 104 is of an elastomeric material that allows the diameter of the tubular portion 104 to change as a medical device is inserted or removed from within the lumen 112. The elastomeric material enables the tubular portion 104 to expand/contract or deform/reform, while maintaining sufficient column stiffness or strength so that the introducer sheath 100 can be inserted into the body lumen. In one configuration, the elastomeric material can be any of those described herein and such others as would be identified by one skilled in the art in light of the teaching contained herein.

Optionally, the tubular portion 104 may also be configured to expand to a certain diameter at which point the tubular portion 104 is further configured to split or separate into one or more portions to accommodate other medical devices, such as, but not limited to vessel closure devices, as will be described in more detail hereinafter.

Generally, each of the hub portion 102 and the tubular portion 104 can have at least a portion of which that is generally cylindrical in nature. Although portions 102 and 104 can have generally cylindrical portions, other cross-sectional configurations are possible, such as but not limited to, oval, polygonal, elliptical, or other cross-sectional configurations usable for a medical device that is insertable into a body lumen.

As previously described herein, the introducer sheath 100 may be formed through an injection molding process. In an injection molding process, the hub portion 102 and the elongated tubular portion 104 are generally formed as a unitary member. Benefits of forming the introducer sheath 100 as a unitary member include reduced costs, increased accuracy of part dimensions (i.e. dimension control) due to lack of assembly, alignment between the lumen 112 of the tubular portion 104 and the lumen 110 of the hub portion 102, and the balancing of mechanical properties across the entire sheath 100 or of any particular portion or member of the sheath 100. The thickness of the walls of the hub portion 102 and/or of the tubular portion 104 can also be controlled and varied as desired during the injection molding process.

Although reference is made herein to fabrication of the introducer sheath 100 through use of injection molding techniques, one skilled in the art will appreciate that various other techniques can be used. For instance, the introducer sheath can be fabricated using milling, grinding, laser treatment, etching, or other techniques to form the introducer from a piece of material. Further, other techniques or methods can include those techniques used by those skilled in the art to fabricate medical devices.

With continued reference to FIG. 1, disposed within the hub portion 102 is a flexible valve member 122 disposed in the hub portion 102. The valve member 122 may be integrally formed into the hub portion 102 during the molding process of the sheath 100, or may be inserted after the sheath 100 is integrally formed. For instance, the hub portion 102 can included a receiving feature 126, such as a groove or channel, to receive the valve member 122. The cooperation between the receiving feature 126 and the valve member 122 result in a sealed hub portion 102. Stated another way, the valve member 122 is self sealing once it is inserted into the hub portion 102 to prevent fluid escaping from the body lumen.

The valve member 122 can be a seal and can have a variety of different configurations to seal the sheath 100. The valve member 122, for example, may have an elastomeric body, such as silicone rubber, with at least one slit and/or other collapsible openings formed therein to allow selective insertion and removal of medical devices or instruments, such as guide wires, catheters, balloon pumps, and other such devices. At the same time, the material and/or structure of the valve member 122 maintains a fluid tight seal around the medical devices or instruments. Thus, blood or other bodily fluids are prevented from leaking out, and unwanted air is prevented from entering into the body.

It will be understood that the valve member 122 can be mounted or coupled to the hub portion 102 in a number of other manners to achieve the sealed configuration for the hub portion 102. For instance, the valve member 122 can be retained with a retaining cap (not shown) disposed adjacent the proximal end of the hub portion 102. In still another configuration, one or more flexible valves or valve members can be mounted within or to the proximal end 116 of the hub portion 102 through the use of one or more retaining caps, rings, or members know to those skilled in the art. Although illustrated as a single member, the valve member 122 can be formed of multiple parts to provide the desired fluid sealing capabilities. Exemplary flexible membranes or valve members which can be utilized with the present invention are shown in U.S. Pat. Nos. 4,798,594, 5,176,652, and 5,453,095 the entireties of which are herein incorporated by reference.

More generally, the introducer sheath can have a configuration similar to the introducer sheath disclosed in U.S. Provisional Patent Application Ser. Nos. 60/659,602, filed Jun. 30, 2005, and entitled “Introducer Sheath”; and 60/695,464, filed Jun. 30, 2005, and entitled “Modular Introducer Sheath; and co-pending U.S. patent application Ser. No. ______, filed Jun. 28, 2006, and entitled “Modular Introducer and Exchange Sheath,” (Attorney Docket No. 16497.12.1) and ______, filed Jun. 28, 2006, and entitled “Introducer Sheath”) (Attorney Docket No. 16497.13.1), the disclosures of which are herein incorporated by reference. As such, the valve member 122 can be mounted in the hub portion 102 and the tubular portion 104 can have a similar configuration to the tubular member to the introducer sheath described in the above-identified applications.

FIG. 1 also illustrates an optional port member 124, such as a luer port/fitting, which may be formed on the hub portion 102. The port member 124 may function as a fluid port for the sheath 100. Fluid (e.g., blood, antibiotics, plasma, saline, etc.) can thus be introduced and/or extracted through the fluid port 124. The port member 124 may also be optionally configured to align and/or selectively lock any device (e.g., a vessel closure device, a catheter) used in conjunction with the sheath 100.

FIGS. 2-5 illustrate various configurations of the tubular portion of the introducer sheath of the present invention. During the insertion/extraction of a medical device or instrument, the tubular portion can deform/form or expand/contract as needed. Thus, the cross sectional area of the tubular portion may change during a medical procedure. In one example, the tubular portion can expand in diameter from a first position to a second position having a diameter greater than the first position as a medical device is either withdrawn or inserted therethrough. The tubular portion can also return to or substantially to the first position following withdrawal or insertion. The illustrated configurations of the tubular portion each have a sheath portion and an elastic portion to provide the desired elasticity, stiffness, or strength. The sheath portion and the elastic portion can be formed from different materials as illustrated in FIGS. 2-4 discussed below. Alternatively, and as illustrated in FIG. 5, the tubular portion can be fabricated from a single material, with the elastic portions being defined through the formation of lumens in the tubular portion. The inclusion of lumens or of elastomeric materials in the formation of an introducer sheath enable the sheath to deform/form or expand/contract as described herein.

Although various features are illustrated in each Figure, any of the features in a particular Figure can be combined with features illustrated in another Figure. Further, the sheath portion and/or elastic portion are examples of one structure capable of performing the function of means for expanding a tubular portion to accommodate the insertion or removal of a medical device.

Turning now to FIG. 2, illustrated is a tubular portion, identified by reference numeral 200, which can be used with the introducer sheath of the present invention, i.e., can function as the tubular portion 104 of FIG. 1, i.e., function to expand/contract or deform/reform to enable withdrawal of a medical device that may have enlarged in diameter during use. The tubular portion 200 can include at least one sheath portion 204 and at least one elastic portion 202. The sheath portion 204 is typically formed of a first material and the elastic portion 202 is often formed of a second material. In FIG. 2, the sheath portion 204 can be formed in strips that run along the length of the tubular portion 200 from the distal end 220 to the proximal end 218, although the strips may have a shorter length. In some embodiments, the strips extend into the distal end 114 (FIG. 1) of the hub portion 102 (FIG. 1). The elastic portion 202 can be formed in strips in this embodiment such that each strip of the elastic portion 202 is adjacent to strips of the sheath portion 204.

The elastic portion 202 can be an elastomer that is bonded to the sheath portion 204. In FIG. 2, each strip of the elastic portion 202 is bonded on each side to adjacent strips of the sheath portion 204. The elastic portion 202 enables the tubular portion 200 to expand or deform such that the interior diameter or cross-sectional area of the lumen can change or increase. The diameter or cross sectional area of the lumen 212 can expand in certain locations and is not required to expand along the entire length of the tubular portion 200. Further, different portions of the tubular portion 200 may expand at different rates and/or at different times. The actual expansion of the tubular portion 200 can depend on a particular medical device that is inserted or withdrawn and/or the material used to form the tubular portion 200. The sheath portion 204 can be selected to ensure that the lumen does not collapse when the tubular portion 200 is in a first or normal or unstressed position and to provide stiffness or rigidity to the tubular portion 200. Thus, the sheath portion 204 provides rigidity, flexibility, and the like or any combination thereof In some embodiments, the sheath portion 204 may also provide some elasticity to the tubular portion 200. Typically, however, the elastic portion 202 has more elasticity than the sheath portion 204.

FIG. 2 also illustrates an optional entry portion 228 to the lumen 212 of the tubular portion 200. The entry portion 228 can be shaped so as to facilitate entry of any device that is entering the tubular portion 200 through the entry portion 228. The entry portion 228 can be formed when the tapered portion 206 is formed and the slope of the tapered portion 206 may be optionally altered to accommodate the entry portion 228. By shaping the entry portion 228, any device being withdrawn can more easily enter the lumen 212 of the tubular portion 200. In one embodiment, the entry portion 228 is concave and the edges at the distal end of the tubular portion 200 are smoothed. In other configurations, the entry portion 228 can be generally curved, smooth, or other configuration to aid with withdrawal of a medical device into the lumen 212.

The elongated tubular portion 200 can include an outer wall 208 and an inner wall 210 thereby defining a wall and a thickness of the wall. As with the lumen 212 (FIG. 1), the lumen 212 extends along the length of the tubular portion 200. The width or diameter or cross sectional area of the lumen 212 can vary and may depend on intended use of the sheath 100. More particularly in this embodiment, the width or diameter or cross sectional area of the lumen 212 can vary or expand and contract during use as the elastic portion 202 changes shape, such as stretching and contracting. Because the hub portion 102 (FIG. 1) and the tubular portion 200 are integrally formed in one configuration, the lumen 212 of the tubular portion 200 remains aligned with the lumen 110 (FIG. 1) of the hub portion 102 (FIG. 1) even though the lumen 212 expands, contracts, deforms, or reforms. It is contemplated that the wall thickness along the length of the elongated tubular portion 200 can be varied to vary mechanical properties of the sheath (stiffness, kink resistance, column strength, etc.).

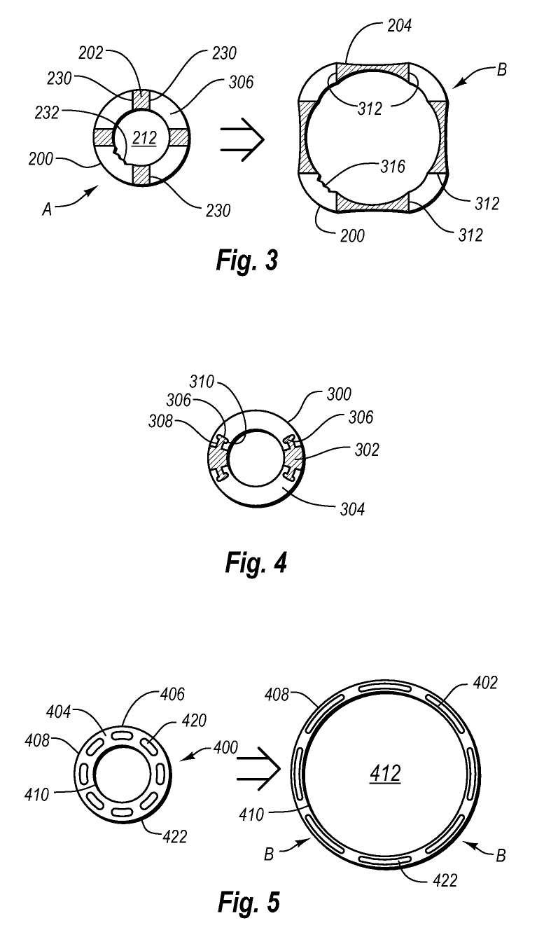

FIG. 3 illustrates a cross section of the tubular portion 200 of the introducer sheath as it moves from first, normal, or unstressed position to a second, expanded, or stressed position of the tubular portion 200. In the first position, identified by reference letter A, the elastic portion 202 of the tubular portion 200 is in a contracted or relaxed state and is bonded to the material of the sheath portion 204 at the bond points 230. The sheath portion 204 can be typically formed from a material such that the lumen 212 of the tubular portion 200 does not seal or close or collapse in the first position, and/or to provide stiffness or flexibility to the tubular portion 200.

In the second position, identified by reference letter B, the elastic portion 202 is expanded while the sheath portion 204 has not expanded (or has expanded less than the elastic portion 202) but is still bonded to the material at the bonds 230. In one embodiment, the sheath portion 204 may have some elasticity, but is generally configured to have less elasticity than the elastic portion 202. The bond strength at the bond 230 may be selected to permit the expansion of the tubular portion 200 to a predetermined diameter or by a predetermined amount. When that diameter or amount is exceeded, the tubular portion 200 may split at the bonds 230 or at another location.

In some embodiments, a geometric pattern 232 is formed on the inner wall 210 or inner surface of the tubular portion 200, such as over all or at least one portion of the inner wall 210 or inner surface. Further, the geometric pattern 232 can be formed in or on the elastic portion 202 and/or the sheath portion 204. This geometric patter 232 can be used to impart certain desirable mechanical properties to the tubular portion 200, such as, but not limited to, stiffness, strength, kink resistance, or flexibility to the tubular portion 200.

Various structures and configurations of the geometric pattern 232 can be used to provide the desired mechanical properties. For instance, in the illustrated configuration, the geometric pattern 232 is formed on one portion or surface of the inner wall 210 of the sheath portion 204 though use of one or more grooves or recesses. The illustrated geometric pattern 232 can represent a plurality of longitudinal grooves extending along an axis parallel to the longitudinal axis of the introducer sheath in a generally uniformly distributed pattern. In other configurations, however, the geometric pattern 232 can be unevenly distributed or a combination of uniformly and unevenly distributed over all or a portion of the inner wall 210 of the tubular portion 200. Further, the location of the grooves need not be parallel to the longitudinal axis of the introducer sheath, but can be transverse to such an axis and/or at any other angular orientation relative to the longitudinal axis.

It shall be understood that the pattern 232 as shown in FIG. 3 should be considered exemplary and not limiting in any manner. It is contemplated that additional styles and types of patterns may be utilized in accordance with the present invention. For example, the pattern 232 may be a sinusoidal pattern disposed about the inner radius of the tubular portion 200. Alternatively, the pattern 232 may be configured to run along a different axis than one parallel to the longitudinal axis of the introducer sheath. For example, the pattern 232 may be formed as a spiral. The pattern 232 may also only extend partially along the length of the tubular portion 200.

Further, the pattern 232 can extend along the length of the tubular portion 200 from the proximal end 220 to the distal end 218 or along a portion of the length of the tubular portion 200. The pattern 232, or any portion thereof, may terminate prior to the proximal end 220 of the tubular portion 200 or extend partially into the hub portion 102 (FIG. 1). The pattern 232 may also be a separation line, such as a pre-scored line. The pattern 232 may be designed to facilitate splitting of at least a portion of the introducer sheath. For example, the introducer sheath may split along all or a portion of the geometric pattern 232 after expanding past a predetermined limit.

FIG. 4 illustrated is a cross section view of another tubular portion of an introducer sheath. This tubular portion 300 can be used with the introducer sheath 100 (FIG. 1) and function to expand/contract or deform/reform to enable withdrawal of a medical device that may have enlarged in diameter during use. The tubular portion 300 has a similar configuration to that of tubular portion 200, and as such the description related to tubular portion 200 also applies to tubular portion 300. As with tubular portion 200, the tubular portion 300 includes at least one elastic portion 302 and at least one sheath portion 304. The elastic portion 302 and the sheath portion 304 are mechanically coupled and/or bonded together to provide additional strength to the connection or coupling between the elastic portion 302 and the sheath portion 304. For instance, in addition to or instead of a thermal or chemical bond between the elastic portion 302 and the sheath portion 304, a mechanical connection is made between the portions 302 and 304 to maintain the coupling or attachment of the elastic portion 302 and the sheath portion 304.

In the illustrated configuration of FIG. 4, the mechanical coupling or connection is facilitated by way of at least one interlocking feature 306 that cooperates and mechanically engages with a corresponding recess or receiving portion of the sheath portion 304. Each interlocking feature 306 can include at least one extension 308, which extends from the main body of the elastic portion 302, and at least on protrusion 310 extending from an end of the extension 308. With the at least one protrusion 310 extending from and being generally transverse to the extension 310, the at least one protrusion 310 aids with preventing detachment of the elastic portion 302 from the sheath portion 304 as the tubular portion 300 extends/contract or deforms/reforms. Although reference is made to the at least one protrusion 310 extending transverse to the at least one extension 308, one skilled in the art will appreciate that the at least one protrusion 310 can extend from the at least one extension 308 are other angular orientations while still being capable of preventing detachment.

The at least one interlocking feature 306 illustrated in FIG. 4 can extend from a proximal end to a distal end of the tubular portion 300 and/or the introducer sheath. It will be understood, however, that the at least one interlocking feature 306 can extend only part way from the distal end toward the proximal end, from the proximal end to the distal end, or at any location along the length of the tubular portion 300. Similarly, although the interlocking feature 306 is illustrated as extending from the elastic portion 302 toward the sheath portion 304, it will be understood that the corresponding recess or receiving portion of the sheath portion 304 can also be considered an interlocking feature. Further, the elastic portion 302 can be configured with the corresponding recess or receiving portion, while the sheath portion 304 includes the at least one extension 308 and/or the at least one protrusion 310.

The interlocking feature 306 of the tubular portion 300 of FIG. 4 can be formed during the manufacturing process of the introducer sheath. For instance, the interlocking feature 306, with the corresponding recess or receiving portion, can be formed during injection molding or during a co-extrusion process of the tubular portion 300 and/or the introducer sheath. Alternatively, the interlocking feature 306 can be formed during manufacture of the elastic portion, such as by injection molding or a co-extrusion process, with the elastic portion being subsequently bonded or coupled to the sheath portion, or vice versa, through thermal bonding, chemical bonding, or other known technique to bond similar or dissimilar medical grade materials.

Turning now to FIG. 5, illustrated is a cross section view of another tubular portion of an introducer sheath. This tubular portion 400 can be used with the introducer sheath 100 (FIG. 1) and function to expand/contract or deform/reform to enable withdrawal of a medical device that may have enlarged in diameter during use.

FIG. 5 illustrates a cross section of the tubular portion 400 of the introducer sheath as it moves from a first, normal, or unstressed position to a second, expanded or stressed position of the tubular portion 400. In the first position, again identified by reference letter A, the tubular portion 400 is in a contracted or relaxed state. The tubular portion 400 is similar to the tubular portion 104 (FIG. 1), but further includes a plurality of lumens 420 disposed at least partially in a wall 422 defined by an outer wall 408 and an inner wall 410 of the tubular portion 400. The region of the tubular portion 400 containing the plurality of lumens 422 has a smaller wall thickness than the remainder of the tubular portion 400. These regions of smaller wall thickness function as elastic portions 402 of the tubular portion 400, while those regions of the wall 422 having no lumens 422 function as the sheath portion 404. Stated another way, the inclusion of the plurality of lumens 422 provides elasticity, expandability, or deformability to the tubular member 400 at the elastic portions 402. The number of lumens 422 in the tubular portion 400 can vary based upon the degree of flexibility desired for the tubular portion 400. Further the particular size, cross sectional shape, and/or ratio of lumen cross-sectional area to area of the wall can be varied to obtain different column strength, stiffness, kink resistance, elasticity, deformability, or other desirable mechanical properties or characteristics of the tubular member 400.

In the expanded position, identified by reference letter B, the at least one lumen 422 enables the relatively thinner wall portions of the tubular member 400 to stretch, thereby increasing the cross sectional area or shape of the tubular portion 400. After expansion, the tubular portion 400 can return to the first position.

Generally, by forming the tubular portion as a composite member using materials having the desired elastic properties, whether or not the tubular portion includes at least one lumen to increase the elasticity, expandability, or deformability of the tubular portion, mechanical properties of the tubular portion may be adjusted as desired. For example, if it is desirable to produce a sheath that is expandable during use, an elastomeric material can be selected along with another material having lower elastic properties. Forming a sheath using these materials, particularly in the tubular portion, provides the sheath with the ability to expand when subject to an applied force. As discussed herein, the configuration of the two or more materials in the sheath can vary and may depend on use. For example, one of the materials may be selected to stiffen the overall tubular portion, prevent kinking in the tubular portion, and the like while the other material is selected based on an elastic property. The bond between the first and second materials can be adjusted to facilitate expansion of the sheath at an appropriate time or for other reasons.

In addition, the use of a geometric pattern can also be combined with the expandability of the sheath. The geometric pattern formed on the inner wall may be used to assist in splitting the sheath during use at an appropriate time, such as when the diameter exceeds a predetermined limit during expansion of the tubular member.

As described above, two or more materials may be utilized to form the sheath in accordance with the present invention. For example, a different material maybe utilized to form the hub portion and one or more materials may be utilized to form the tubular portion of the sheath. Again, the selection of materials may depend on the end use of the sheath, properties of medical devices used with the sheath, and the like or any combination thereof Although the present invention has been shown and described in accordance with specific embodiments these should not be considered limiting in any manner. For example, multiple materials may be utilized to form a unitary sheath in accordance with the present invention, wherein multiple injection molding processes are performed simultaneously or in stages to form the unitary sheath in accordance with the present invention.

Embodiments of the introducer sheath described herein can be used in various medical procedures. In one example, a medical procedure often begins by introducing a sheath into body lumen, such as a patient's vasculature. After the sheath is properly introduced, a first medical device can be introduced through the sheath. During introduction of the first medical device, the sheath or at least the tubular portion of the sheath may expand to accommodate a size of the first medical device.

After the first medical device has been introduced, the medical procedure may be performed. During this procedure, in one example, the size of the first medical device may change. During withdrawal of the first medical device, at least the tubular portion of the sheath can expands or deform accommodate the increased size of the first medical device. The expansion or change in cross sectional area can occur at different locations of the sheath or of the tubular portion as the first medical device is withdrawn.

After the first medical device is withdrawn, a second medical device, such as a vessel closure device, stent delivery device, or other medical device, can be introduced through the sheath. This newly inserted medical device can be used without prior insertion of another introducer sheath. In the case of the vessel closure device, the vessel closure device can be placed in the desired location relative to the vessel wall and the introducer sheath removed before, during, or part of the vessel sealing process.

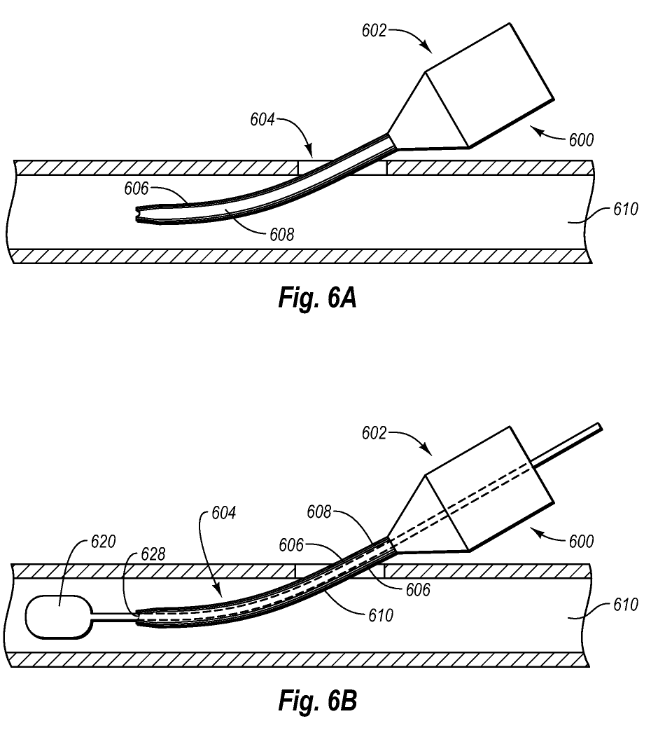

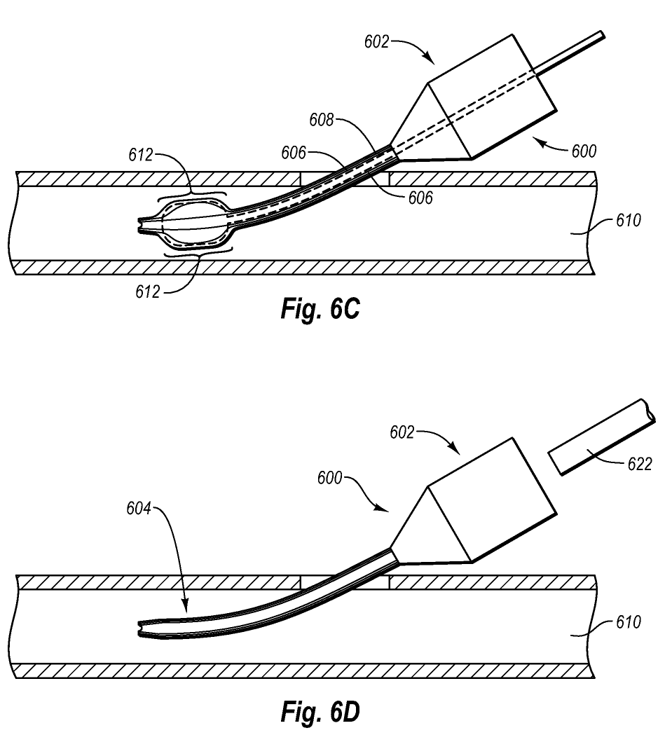

The above-described process is illustrated in more detailed with reference to FIGS. 6A-6D. FIGS. 6A-6D illustrate an example of one configuration of an expandable introducer sheath during use in a medical procedure. A sheath 600, which can be any of the introducer sheaths previously described with respect to FIGS. 1-5, can be inserted into a vessel or vasculature 610 of a patient. In this example, the tubular portion 604 of the sheath 600 is formed of a first elastomeric material 606 and a second material 608. With the introducer sheath 600 in place one or more medical devices or instruments can be passed therethrough, such as through the lumens of the hub portion 602 and the tubular portion 604, to gain access to the vasculature 610 and more particular to a treatment site.

In one configuration, and with reference to FIG. 6B, a medical device, such as, but not limited to, a dilation balloon or an intra-aortic balloon pump, identified by reference numeral 620, can be passed through the hub portion 602. During use of the medical device 620, the outside diameter of the medical device 420 increases in size from when it was originally introduced into the vasculature 610 by way of the sheath 600. The structure and function of the tubular portion 604 and/or the introducer sheath 600 can, however, accommodate such a change and eliminates the need to remove the introducer sheath 600 with the medical device 620.

With continued reference to FIG. 6B, once the medical procedure is complete, the medical device 620 can be withdrawn until the medical device 620 contacts the distal end of the introducer sheath 600 and/or the entry portion 628 that facilitates entry of the medical device 620 back through the sheath 616. As the medical device 620 is withdrawn, its size introduces a force that causes the tubular portion 604 to expand as the first elastomeric material 606 flexes, expands, or deforms accommodate the pump 604, as illustrated in FIG. 6C.

As the medical device 620 is withdrawn through the tubular portion 604, regions of the first elastomeric material 606 expand, such as in region 612, such that a cross sectional area of the lumen of the sheath 600 has increased at least at this location. The medical device 620 can therefore be withdrawn without splitting the sheath 600 or without having to remove the sheath 600 from the vasculature 610 during removal of the medical device 620.

After the medical device 620 is withdrawn, FIG. 6D illustrates that another medical device 422, such as a vessel closure device, a stent delivery device, or other medical device, can be introduced into the vasculature 620 via the sheath 600. Without the expansion capability enabled by the embodiments disclosed herein, the sheath would have to be removed earlier than desired, which could preclude use of other medical devices, such as, but not limited to, the vessel closure device.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An introducer sheath comprising:
a hub portion having a lumen formed therein;
a tubular portion extending from the hub portion, the tubular portion comprising at least one elastic portion elastically deformable to increase a cross sectional area of the tubular portion.

2. The introducer sheath of claim 1, the hub portion further comprising a valve member.

3. The introducer sheath of claim 1, wherein the hub portion and the tubular portion are formed as a unitary member using one of an injection molding process or a co-extrusion process such that a distal end of the hub portion transitions to a proximate end of the tubular portion, the introducer sheath further comprising a strain relief at the transition.

4. The introducer sheath of claim 1, wherein the tubular portion further includes a sheath portion comprising a first material, and the elastic portion comprising a second material including an elastomer.

5. The introducer sheath of claim 4, wherein the elastic portion comprises a plurality of strips and the sheath portion comprises a plurality of strips, each strip of the elastic portion being bonded to adjacent strips of the sheath portion.

6. The introducer sheath of claim 5, each strip of the elastic portion includes an interlocking feature to attach with adjacent strips of the tubular portion.

7. The introducer sheath of claim 1, wherein the tubular portion further comprises an entry portion at a distal end, the entry portion facilitating entry of a medical device that is being withdrawn back through the tubular portion, wherein the tubular portion expands to accommodate the medical device without splitting the tubular member.

8. The introducer sheath of claim 1, the elastic portion comprising a plurality of lumens formed in a wall of the tubular portion, the plurality of lumens providing elasticity to the sheath portion.

9. The introducer sheath of claim 1, an inner wall of the tubular member including a geometric pattern.

10. The introducer sheath of claim 1, further comprising a fluid port extending from the hub portion.

11. An introducer sheath, comprising:
a hub portion;
a tubular portion extending from the hub portion, the tubular portion comprising a first material and a second material, wherein the second material enables the tubular member to expand from a first position to a second position.

12. The introducer sheath of claim 11, wherein the hub portion further comprises a valve member that seals the hub portion.

13. The introducer sheath of claim 12, wherein the valve member further comprises an elastomeric body having at least one slit formed therein to allow selective insertion and removal of a medical device while maintaining a fluid tight seal around the medical device.

14. The introducer sheath of claim 11, wherein the hub portion further comprises a fluid port to facilitate the introduction or removal of fluid from vasculature.

15. The introducer sheath of claim 14, wherein the fluid port comprises an alignment mechanism that aligns a medical device introduced through the sheath.

16. The introducer sheath of claim 11, wherein the tubular portion further comprises:
a plurality of first strips formed from the first material; and
a plurality of second strips formed from the second material, wherein each first strip is bonded to at least one second strip and wherein the plurality of first strips provide rigidity to prevent a lumen of the tubular portion from collapsing.

17. The introducer sheath of claim 11, further comprising a strain relief formed at a transition between the hub portion and the tubular portion.

18. The introducer sheath of claim 11, wherein the tubular member further comprises a geometric pattern formed on at least a portion of an inner wall of the tubular portion.

19. The introducer sheath of claim 18, wherein the geometric pattern comprises at least one of:
a groove that facilitates splitting of at least the tubular portion after the second material exceeds an expansion point;
a separation line to facilitate splitting of the tubular portion;
a plurality of grooves running parallel to a longitudinal axis of the tubular portion; or
a plurality of grooves that are not parallel to the longitudinal axis.

20. The introducer sheath of claim 11, wherein the tubular portion further comprises a plurality of lumes formed in a wall of the tubular portion, the plurality of lumens providing additional elasticity to the tubular portion.

21. The introducer sheath of claim 11, wherein the tubular portion further comprises a tapered end form at a distal end of the tubular member.

22. The introducer sheath of claim 21, the tapered end further comprising an entry portion that facilitates entry of a medical device back through the tubular portion, wherein the tubular portion expands to accommodate the withdrawal of the medical device.

23. A method for performing a medical procedure, the method comprising:
introducing a sheath into a lumen of a patient;
inserting a first medical device into the lumen through the sheath, wherein a medical procedure is performed with the first medical device and wherein a size of the first medical device increases during the medical procedure; and
extracting the first medical device through the sheath, wherein at least a tubular member of the sheath expands to accommodate the increased size of the first medical device.

24. The method of claim 23, further comprising inserting a second medical device through the sheath

25. The method of claim 24, wherein inserting a second medical device through the sheath further comprises:
introducing a vessel closure device through the sheath; and
closing the lumen of the patient with the vessel closure device.

26. The method of claim 23, wherein the sheath comprises a tubular portion extending from the hub portion, the tubular portion comprising at least one elastic portion elastically deformable to increase a cross sectional area of the tubular portion.
